(19) United States
(12) Patent Application Publication
(10) Pub. No.: US 2025/0113833 A1
(43) Pub. Date: Apr. 10, 2025

(54) FOOD, COOKING APPLIANCE FOR PREPARING THE FOOD, AND METHOD FOR CONTROLLING THE COOKING APPLIANCE

(71) Applicant: BSH Hausgeräte GmbH, Munich (DE)

(72) Inventors: Uwe Has, Unterneukirchen (DE); Barbara Heikel, Traunstein (DE)

(21) Appl. No.: 18/836,397

(22) PCT Filed: Mar. 20, 2023
(86) PCT No.: PCT/EP2023/057002

§ 371 Date: Aug. 7, 2024

(30) Foreign Application Priority Data
Mar. 30, 2022 (DE) 10 2022 203 107.5

(51) Int. Cl. A21D 13/41; F24C 7/08; G01N 33/10

(52) U.S. Cl. CPC A21D 13/41; F24C 7/085; G01N 33/10

(57) ABSTRACT

A food in a form of a semi-ready-made product or a ready-made product includes an indicator substance which is insignificant for a cooking process of the food and which emits a defined signal when a defined preparation progress of the food is reached or in an initial state. The defined signal is detected by a sensor embodied in or on a cooking appliance.

### TECHNICAL FIELD

The present disclosure relates to a food in the form of a (semi-) ready-made product, a cooking appliance for preparing the (semi-) ready-made product as well as a method for controlling the cooking appliance.

### PRIOR ART

There exist cooking appliances, in particular ovens, for preparing various foods, such as cakes or pizza for example, with corresponding sensor-supported automatic modes. If the cooking appliance is operated in the corresponding mode, the cooking appliance is able to recognize, for example when preparing pizza, that the pizza is ready and can be removed from an oven cavity of the cooking appliance. In this context, sensors are used which monitor the air humidity in the oven cavity. For example, when the air humidity is at a maximum, the cooking appliance recognizes that the pizza is ready. For this, however, it is essential that the cooking appliance knows which food/ready-made product is located in the oven cavity. In other words, a pizza program does not necessarily have to function for preparing a cake baked in a tin. The same applies if, despite the dish of “pizza” remaining the same, instead of a pizza according to a standard recipe, a deep-frozen ready-made pizza or another ready-made dish or a semi-ready-made dish, made from a baking mixture or a ready-made pizza dough for example, is to be prepared.

The producers of such pre-prepared semi-ready-made products handle this problem by optimizing their products such that they are fully prepared/cooked, where possible, after a particular time in the cooking appliance when said cooking appliance is set in a particular manner.

Furthermore, the products are represented by the producers, for example on packaging of the products, such that the “ready time” where possible is not determined in a precise manner, but rather can be seen in the form of a time interval/time window which is as long as possible. This happens because the semi-ready-made products are prepared in a wide range of different cooking appliances, each having different cooking characteristics, meaning that a preparation duration can vary in a wide time window. The user therefore has to continuously supervise the cooking appliance and rely on their sensory capabilities, in order to identify the ready time.

For this purpose, DE 10 2004 037 606 A1 discloses a method for determining the progression of baking procedures of an item to be baked in an oven. For this, the method measures the CO₂ content of the atmosphere surrounding the item to be baked. This method has the disadvantage, however, that it is exclusively applicable to baking procedures in which a raising agent is used.

An object of the present disclosure is therefore to eliminate or at least reduce the disadvantages from the prior art.

In particular, an object of the present disclosure is to provide a food in the form of a (semi-) ready-made product, the ready time of which can be determined by a cooking appliance in a reliable manner.

Furthermore, an object of the present disclosure is to provide a food in the form of a (semi-) ready-made product, which can be identified by a cooking appliance.

Furthermore, an object of the present disclosure is to provide a corresponding cooking appliance, which determines the food and/or the ready time thereof, as well as a method for controlling such a cooking appliance.

These objects are achieved, with regard to the food in the form of a semi-ready-made product or a ready-made product, by a food with the features of claim 1 and, with regard to the cooking appliance and control method thereof, by a cooking appliance and method with the features of the further independent claims. Advantageous developments are the subject matter of subclaims in each case.

Specifically, the object is achieved by a food in the form of a semi-ready-made product or a ready-made product with an indicator substance that is insignificant for a cooking process of the food, which emits a defined signal when a defined preparation progress of the food is reached or in an initial state, which signal is provided and embodied to be detected by a sensor embodied in or on a cooking appliance.

In other words, according to the present disclosure the food contains the indicator substance in the form of an additive added to the industrially prepared food, wherein the indicator substance is not necessary for the preparation process of the food, but rather is exclusively used to identify the food and/or to identify the progress to be achieved in the preparation of the food. In other words again, the preparation or the cooking process of the food is independent of the indicator substance. The indicator substance also does not alter characteristics of the food over the temporal course of the cooking process.

The indicator substance emits a detectable item of information in the form of a signal. The signal involves a sign with a particular meaning. In this context, the signal can have any given form, as long as it can be identified in a reliable and unambiguous manner by the sensor embodied on the cooking appliance. In this context, the signal can already be present before the start of the actual cooking process and/or can become active during the cooking process and/or can change over the temporal course of the cooking process.

By embodying the food/the prepared (semi-) ready-made product in this manner, the preparation thereof can take place in a substantially more reliable manner. In particular, the ready time of the food can be reliably determined independently of the kind of food, i.e. independently of whether the food is a cake or a pizza for example. Furthermore, it is possible to use the indicator substance to further differentiate between foods of the same type, of which there are generally countless variants, so that there is no restriction to broad classifications of foods into categories such as pizza or cake. A food of this kind additionally ensures that, independently of variations in appliance operation by the customer, the food is prepared in a reliable manner as intended by the producer. For example, the ready time of a food of this kind can be reliably determined independently of whether the food is introduced into the cooking appliance in a frozen state or in a defrosted state. In general, by way of an indicator substance that is insignificant for the preparation process of the food, the indicator substance can be specifically aligned to the defined preparation progress of the food to be detected, and thus the detection can be improved.

The indicator substance involves a substance or a food additive, which meets the requirements for food safety, in particular is not hazardous to health and is suitable for human consumption. In particular, this also applies for any products caused by a (degradation) reaction of the indicator substance. It is also conceivable, however, that the indicator substance is substantially inedible in a state in which the food is (partially) raw/uncooked, and only becomes consumable/edible by way of the preparation process.

The food is to be understood as a food once it has been removed from an associated (outer) packaging. The indicator substance is explicitly not located on the packaging.

In one aspect, the defined preparation progress/cooking progress can involve a reaching of a particular temperature of the food, in particular by a particular necessary core temperature of the food and/or an in particular full defrosting of the food and/or an occurrence of at least one particular chemical reaction.

In a more abstract sense, the defined preparation progress can also involve a defined loosening of a tissue, a fiber structure and/or a cell framework of the food as well as a denaturing of proteins of the food.

Furthermore, other definitions of the preparation progress are conceivable. Thus, a color, in particular a degree of browning, a moisture content, a consistency, a firmness or the like can also be used as defined preparation progress.

In a further aspect, the defined signal can involve at least one chemical signal, such as a gas and/or an odor substance for example, and/or an optical signal, such as a color change for example, and/or a solidification of the food and/or a change in the shape and/or volume of the food.

In other words, when the defined preparation progress is reached, the indicator substance can release the chemical signal, for example by the indicator substance splitting into multiple products, of which at least one can be present in the form of a gas. Alternatively or additionally, the defined signal can be an optical and/or optically readable signal.

In a further aspect, the indicator substance can involve riboflavin and/or a baking powder.

In other words, riboflavin, also referred to as lactoflavin, vitamin B2 or food additive with E number E101, can be used as indicator substance. Riboflavin fluoresces in an aqueous solution with a yellow-green hue at approx. 565 nm. In other words, riboflavin indicates a yellow-green fluorescence in an aqueous solution when irradiated with a UV light source. Using riboflavin, it is possible to apply at least one pattern and/or identifier to the food, which are easily recognizable under UV light that is invisible per se for the human eye, in particular under black light.

More specifically, it is possible to apply an identifier, for example, a pattern, a geometric shape, an alphanumeric code, a machine-readable code, for example a barcode or a QR code or the like, to the food using riboflavin. This identifier makes it possible to unambiguously identify the food and/or to identify preparation parameters of the food. In the event that the identifier involves an alphanumeric code or a machine-readable code, the preparation parameters for the food can be depicted directly on the food. An identifier of this kind can be detectable by at least one optical sensor, in particular by a camera, in the cooking appliance, optionally with the use of the UV light source.

With the identifier using riboflavin as indicator substance, the indicator substance and thus the signal can be invariable at least over wide ranges of preparation. In this case, the signal is first of all used to identify the food.

Alternatively or additionally, the indicator substance can involve a baking powder or a baking powder mixture.

There exist different baking powders with differing mixtures of various ingredients in each case. Examples of possible ingredients are sodium bicarbonate (baking soda, E500), potassium bicarbonate (E501), disodium hydrogen phosphate (E450a), monocalcium orthophosphate (E341a), tartar or the like.

Baking powder produces CO₂ when combined with moisture, but stops doing so when a certain baking powder-specific (maximum) temperature is reached. In this manner it is possible to detect, for example, when the food in the form of the frozen (semi-) ready-made product is heated during the preparation. CO₂ production begins immediately after defrosting the food that is embodied with the baking powder. CO₂ production ends at the latest when the baking powder-specific temperature is reached. In this manner, the baking powder acts as a temperature sensor for the food, via which it is possible to detect two characteristic times in a progression of the preparation process. The signal that is transmitted is a presence and/or a concentration of CO₂ in the cooking appliance, in particular in the cooking compartment of the cooking appliance. A signal of this kind can be detectable by at least one gas sensor, in particular a CO₂ sensor, in the cooking appliance. In this context, the baking powder has no function for the actual cooking procedure of the food and is used exclusively as an indicator substance.

In a further alternative embodiment, the indicator substance can involve a baking powder with a specific mixing ratio, which only starts to produce gas at an increased temperature (for example at 60° C.). Examples of possible ingredients of a baking powder of this kind are ammonium bicarbonate, potassium carbonate (potash), salt of hartshorn, ammonium carbamate or the like.

An indicator substance of this kind can thus indicate that the food has already approached the target temperature. With an indicator substance of this kind, ammonia can also be released as a signal as part of the cooking process. Alternatively or additionally, a signal of this kind can be detectable by an ammonia sensor in the cooking appliance.

In a further aspect, the indicator substance can be evenly distributed in or on the food.

In other words, the indicator substance can be evenly scattered over a surface of the food and/or incorporated into the food in an evenly distributed manner.

By evenly distributing the indicator substance in this manner, it is possible to ensure a reliable detection of the signal. It is thus possible to ensure, for example, that despite any temperature gradients in the cooking compartment of the cooking appliance, the indicator substance at least partially emits the (in this case temperature-dependent) signal by locally reaching the target temperature of the food.

In a further aspect, the indicator substance can be embodied in and/or on at least a selected region of the food that is identified as especially critical, in particular in relation to a cooking point, during the preparation of the food.

In other words, the indicator substance can be explicitly embodied in the at least one region of the food in which the food cooking thoroughly is of high importance, for example for health reasons. Thus, the at least one region can involve a region which consists of animal product, for example fish or meat. In such a region, it is of particularly great importance that the target temperature of the food is reached in the cooking process. Alternatively or additionally, the region can involve a region that has particular geometric dimensions. For example, it can involve an olive on a pizza, which makes up the thickest point of a pizza topping.

In a further aspect, the indicator substance can be embodied in a deposit in a food, meaning that it is ensured that the signal only becomes active at a certain time.

Furthermore, an object of the present disclosure is to provide a cooking appliance which reliably determines the ready time of the (semi-) ready-made product. The cooking appliance can involve an electric oven, a gas cooker, a cooktop or any other built-in large electric appliance which is suitable for cooking food. Furthermore, the cooking appliance can involve a transportable electric appliance, such as a food processor with cooking function.

The cooking appliance according to the disclosure for preparing a food according to one of the above aspects contains at least one sensor, which is provided and embodied to detect a signal emitted by an indicator substance, which is insignificant for the cooking process of the food, in the food.

In other words, the cooking appliance with the at least one sensor is preferably embodied in an interior of the cooking appliance. The sensor preferably involves a camera, a gas sensor or the like, which is aligned to the signal emitted by the indicator substance of the food. In other words again, the sensory equipment in the cooking appliance is suitable for detecting the signal that the indicator substance generates.

Due to the fact that the sensory equipment of the cooking appliance anticipates signals from the indicator substance that are not given off by the food itself as a result of a cooking process that is natural and thus subject to tolerances, but rather the sensory equipment anticipates signals that are generated specifically for the sensory equipment, it is possible for the recognition of the reaching of a defined preparation progress of the food and/or the identification of the food to take place in a reliable manner.

In one aspect, the cooking appliance can contain an identification unit which identifies the food on the basis of the signal emitted by the indicator substance and sets a cooking program of the cooking appliance on the basis of an identification result output by the identification unit.

In other words, the identification unit of the cooking appliance can evaluate the signals that are detected by the at least one sensor and are emitted by the indicator substance of the food, and can compare the detected signals with a database embodied in the cooking appliance, preferably in the identification unit of the cooking appliance, and identify the food on the basis of the comparison.

Alternatively or additionally, the cooking appliance can be connected to a network/server, in particular a cloud, preferably via a communication module.

The database with which the identification unit compares the detected signals can alternatively or additionally be stored in the cloud.

Once it has identified the food, the identification unit can set parameters of the cooking appliance according to the food identified. Parameters of the cooking appliance can be understood to mean a cooking temperature, a manner of operation (for example top heat and/or bottom heat and/or circulating air and/or a timed sequence of said manners of operation), a cooking duration or the like.

Alternatively or additionally, the identification unit of the cooking appliance can identify the parameters directly from the signals given off by the indicator substance. In other words, the parameters can be applied to the food in a machine-readable manner (for example alphanumerically, in a barcode or in a QR code) using the indicator substance. The at least one sensor can detect the parameters in this case and the identification unit can perform the corresponding settings on the cooking appliance.

By way of an identification unit of this kind, it can be ensured that the correct parameters are set for the cooking appliance for preparing the food and the desired cooking result is achieved in a reliable manner. Additionally, operation becomes considerably more convenient or is simplified for a user of the cooking appliance, as it is possible to dispense with manually entering parameters.

In a further aspect, the cooking appliance can contain an evaluation unit which, on the basis of the signal emitted by the indicator substance, determines whether the food in the cooking appliance is fully prepared and/or makes a prediction as to when the food in the cooking appliance will be fully prepared.

In other words, the evaluation unit of the cooking appliance can evaluate the signals that are detected by the at least one sensor and are emitted by the indicator substance of the food, and can compare the detected signals with a database embodied in the cooking appliance, preferably in the evaluation unit of the cooking appliance, and identify whether the food has been fully prepared on the basis of the comparison. Alternatively or additionally, the evaluation unit can determine an expected ready time of the food based on the comparison.

The database with which the evaluation unit compares the signals detected by the sensor can alternatively or additionally be stored in the cloud.

Optionally, when the evaluation unit determines that the food has been fully prepared, the cooking appliance can conclude the preparation and/or output a notification alarm and/or switch to a keep-warm operating mode of the cooking appliance.

Further optionally, the cooking appliance can output the predicted ready time to the user and/or output a countdown until the predicted ready time to the user.

Further optionally, the evaluation unit can recognize that a food that was originally deep-frozen has already defrosted and adapt the preparation accordingly and/or output a corresponding notification to the user.

By way of an evaluation unit of this kind, it is possible to ensure that the optimal ready time for the corresponding food is achieved in a reliable manner, without the user having to rely on vague indications on the packaging or their personal sensory capabilities. Additionally, by concluding the preparation automatically, it is possible to prevent the food from burning in the cooking appliance if it is forgotten by the user, which represents a safety risk.

In a further aspect, the identification unit of the cooking appliance can align the parameters for preparing the food to the corresponding cooking appliance on an individual basis. Thus, for example, in a cooking appliance with a particularly large cooking compartment, the preparation time can be extended due to a longer heating-up procedure.

In a further aspect, the cooking appliance can transmit the identified type of food to the network. In this manner, the producer can collect valuable data on the actual use of the cooking appliance.

In a further aspect, the parameters for the preparation can be optimized on the network side by transmitting suitable or more suitable parameters.

In a further aspect, information regarding the actual preparation process can be transmitted to the network. Thus, for example, it is possible to transmit when users regularly manually interrupt the preparation of a specific food before the actual ascertained preparation time. In this manner, it is possible to reliably ascertain errors or possibilities for optimization in relation to the preparation parameters.

The data transmitted to the network can be transmitted both to the producer of the food and to the producer of the cooking appliance.

The object of the present disclosure is also achieved by a method for controlling a cooking appliance with the steps:
identification of the food on the basis of signals emitted by an indicator substance of the food, wherein the indicator substance in particular can be insignificant for the preparation of the food;
setting a cooking program;
detecting and/or predicting an end of a cooking procedure on the basis of signals emitted by an indicator substance of the food;
displaying the previously ascertained data and/or transmitting the data to a server.

Individual steps of the method can be optional here. It is thus possible to dispense with the detection or prediction of the end of the cooking procedure, for example, if a preparation duration is specified in the cooking program that is set.

Alternatively, it is possible to dispense with an automatic identification of the food, if the cooking program is set by a user.

In one aspect, the indicator substance for the identification of the food can involve a first indicator substance, and the indicator substance for the detection and/or prediction of the end of the cooking procedure can involve a second indicator substance that differs from the first indicator substance. Alternatively, it is conceivable that the same indicator substance is used both for the identification and for the detection and/or prediction of the end of the cooking procedure.

Furthermore, the object of the present disclosure is achieved by a system consisting of the food in the form of a semi-ready-made product or a ready-made product according to one of the above aspects and a cooking appliance according to one of the above aspects, wherein the sensory equipment in the cooking appliance is aligned to the indicator substance or to the signals that the indicator substance emits.

Additionally, the object of the present disclosure is achieved by a use of a preparation progress-dependent indicator substance in a food, which is insignificant for a cooking process of the food, as a detectable signal for detecting a preparation progress of the food.

### BRIEF DESCRIPTION OF THE FIGURES

FIG. 1 discloses a food in a first embodiment according to the present disclosure;

FIG. 2 discloses the food in a second embodiment according to the present disclosure;

FIG. 3 discloses a cooking appliance according to the present disclosure;

FIG. 4 illustrates a method for preparing a food in a flow diagram.

### DESCRIPTION OF THE EXEMPLARY EMBODIMENTS

Exemplary embodiments of the present disclosure are described below on the basis of the associated figures.

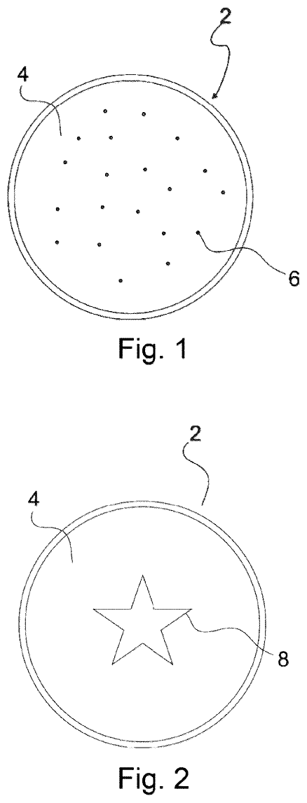

FIG. 1 shows a food in the form of a pizza 2 in a first exemplary embodiment. The pizza 2 involves a commercially available (semi-) ready-made product, which can be fully prepared in a cooking appliance. On a top side of the pizza 2, which corresponds to a topping side 4 of the pizza 2, in addition to toppings (not shown) a first indicator substance 6 is distributed substantially evenly over the topping side 4. The first indicator substance 6 is insignificant for a cooking process of the pizza 2 and does not change the characteristics of the pizza 2 beyond the cooking process. When the pizza 2 is heated during the preparation, the first indicator substance 6 emits a detectable signal, for example in the form of a gas, at defined temperatures, for example at a temperature that corresponds to the temperature of the pizza 2 when fully prepared. The emitting of the detectable signal is directly linked to the defined temperature being reached. Alternatively or additionally, the first indicator substance 6 can also be incorporated into a base/dough of the pizza 2 and/or incorporated into the toppings of the pizza 2, for example into the sauce.

FIG. 2 shows the food in the form of the pizza 2 in a second exemplary embodiment. On the top side of the pizza 2, which corresponds to the topping side 4 of the pizza, in addition to the toppings (not shown) a second indicator substance 8 is applied in an optically readable pattern. In the exemplary embodiment shown here, the optically readable pattern involves a star. The pizza 2 can be identified on the basis of the pattern. In this context, on the one hand it is possible for the food, pizza 2, to be characterized in general via the optically readable pattern, via the star here, while on the other hand it is also possible for the food to involve the pizza 2 of a particular producer and/or the pizza 2 with a predetermined topping. The second indicator substance 8 is invisible to the human eye in the second exemplary embodiment. The second indicator substance 8 is riboflavin, for example, which fluoresces yellow-green under UV light. Alternatively, the second indicator substance 8 can also be a substance that is visible to the human eye.

The second indicator substance 8 does not change beyond the cooking process of the pizza 2 in the second exemplary embodiment. It is also possible to conceive embodiments in which on the one hand a third indicator substance is applied to the food in an optically detectable pattern, and on the other hand a further signal is emitted as a function of a cooking progress of the food.

Furthermore, it is possible to conceive a food which contains both the first indicator substance 6 and the second indicator substance 8.

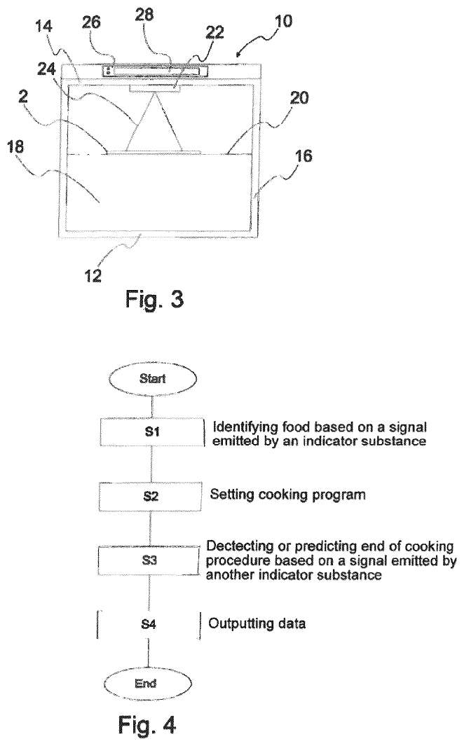

FIG. 3 shows a cooking appliance in the form of an oven 10. The oven 10 contains a muffle with a bottom 12, an upper part 14 and a wall 16 connecting the bottom 12 and the upper part 14. The muffle can be closed off by a door (not shown) hinged on the muffle. It is possible to insert a carrier for food to be cooked, for example in the form of a baking sheet 20, into a cooking compartment 18 embodied by the muffle and the door. The baking sheet 20 is provided and embodied to accommodate the food for preparation. In the embodiment shown here, the food in the form of the pizza 2 is arranged on the baking sheet 20. Embodied on the upper part 14 is a sensor 22, which detects the first indicator substance 6 and/or the second indicator substance 8 on the pizza 2. The sensor 22 and the first indicator substance 6 and/or the second indicator substance 8 are aligned to one another. To be more precise, the sensor 22 is provided and embodied to detect a signal 24, which is emitted by the first indicator substance 6 and/or the second indicator substance 8 on the pizza 2.

The oven 10 furthermore contains a control module 26 with an identification unit (not shown), which identifies the food (in the form of the pizza 2) on the basis of the signal 24. On the basis of an identification result of the identification unit, the control module 26 sets preparation parameters for the oven 10 that are aligned to the pizza 2. Furthermore, the identification unit outputs the recognized food via a screen 28.

The control module 26 of the oven 10 is furthermore embodied with an evaluation unit (not shown). On the basis of the signal 24 detected by the sensor 22, the evaluation unit ascertains whether the pizza 2 has been fully prepared in the oven 10 and/or makes a prediction as to when the pizza 2 will be fully prepared in the oven 10. An evaluation result is output by the evaluation unit via the screen 28.

The control module 26 further contains a communication module (not shown). Via the communication module, (updated) preparation parameters are transferred to the control module from an external server. Additionally, the communication module sends data, for example in the form of usage data, to the server.

Alternatively or additionally, the preparation parameters are adapted over a temporal progression of the preparation as a function of the signal, up to the oven 10 being switched off when the evaluation unit ascertains that the pizza 2 has been fully prepared.

In alternative embodiments, the control module 26 is in each case only embodied with the evaluation unit or the identification unit.

FIG. 4 shows a flow diagram which illustrates a method for controlling the oven 10.

In a first step S1, the food is identified on the basis of the signal 24 emitted by the second indicator substance 8 embodied in and/or on the food, by means of the sensor 22. The food recognized is output via the screen 28.

In a second step S2, the cooking program is set according to the food ascertained in the first step S1 (and is output via the screen 28).

In a third step S3, following the second step S2, an end of the cooking procedure of the food is detected and/or predicted on the basis of signals 24 that are emitted by the first indicator substance 6 of the food and are recorded by the sensor 22.

In a fourth step S4, the data ascertained in the third step S3 is output via the screen 28 and/or transmitted to the server via the communication module.

### LIST OF REFERENCE CHARACTERS

2 pizza
4 topping side
6 first indicator substance
8 second indicator substance
10 oven
12 bottom
14 upper part
16 wall
18 cooking compartment
20 baking sheet
22 sensor
24 signal
26 control module
28 screen

1-10. (canceled)

11. A food in a form of a semi-ready-made product or a ready-made product, the food comprising an indicator substance which is insignificant for a cooking process of the food and which emits a defined signal when a defined preparation progress of the food is reached or in an initial state, said defined signal being detected by a sensor embodied in or on a cooking appliance.

12. The food of claim 11, wherein the defined preparation progress involves a reaching of a particular temperature of the food and/or a defrosting of the food and/or an occurrence of particular chemical reaction.

13. The food of claim 11, wherein the defined signal comprises a chemical signal comprising gas and/or an odor substance, and/or an optical signal comprising a color change, and/or a solidification of the food and/or a change in a shape and/or volume of the food.

14. The food of claim 11, wherein the indicator substance involves riboflavin and/or a baking powder.

15. The food of claim 11, wherein the indicator substance is evenly distributed in or on the food.

16. The food of claim 11, wherein the indicator substance is embodied in and/or on a selected region of the food that is identified as critical, in particular in relation to a cooking point, during a preparation of the food.

17. A cooking appliance for preparing a food, the cooking appliance comprising:
an indicator substance provided in the food and being insignificant for a cooking process of the food; and
a sensor designed to detect a signal emitted by the indicator substance.

18. The cooking appliance of claim 17, further comprising an identification unit designed to identify the food based on the signal emitted by the indicator substance and to set a cooking program of the cooking appliance based on an identification result output by the identification unit.

19. The cooking appliance of claim 17, further comprising an evaluation unit designed to determine based on the signal emitted by the indicator substance as to whether the food in the cooking appliance is fully prepared and/or to predict as to when the food in the cooking appliance will be fully prepared.

20. The cooking appliance of claim 17, wherein the signal comprises a chemical signal comprising gas and/or an odor substance, and/or an optical signal comprising a color change, and/or a solidification of the food and/or a change in a shape and/or volume of the food.

21. The cooking appliance of claim 17, wherein the indicator substance comprises riboflavin and/or a baking powder.

22. The cooking appliance of claim 17, wherein the indicator substance is evenly distributed in or on the food.

23. The cooking appliance of claim 17, wherein the indicator substance is embodied in and/or on a selected region of the food that is identified as critical, in particular in relation to a cooking point, during a preparation of the food.

24. A method for controlling a cooking appliance, the method comprising:
identifying a food based on a signal emitted by an indicator substance of the food;
setting a cooking program;
recording and/or predicting an end of a cooking procedure of the food in response to a signal emitted by another indicator substance of the food; and
displaying previously ascertained data and/or transmitting the data to a server.

25. The method of claim 24, further comprising:
identifying the food based on the signal emitted by the indicator substance to generate an identification result; and
setting a cooking program of the cooking appliance based on the identification result.

26. The method of claim 24, further comprising determining based on the signal emitted by the another indicator substance as to whether the food in the cooking appliance is fully prepared and/or to predicting as to when the food in the cooking appliance will be fully prepared.

27. The method of claim 24, wherein the signal emitted by the indicator substance and/or the another indicator substance comprises a chemical signal comprising gas and/or an odor substance, and/or an optical signal comprising a color change, and/or a solidification of the food and/or a change in a shape and/or volume of the food.

28. The method of claim 24, wherein the indicator substance and the another indicator substance each comprises riboflavin and/or a baking powder.

29. The method of claim 24, further comprising evenly distributing the indicator substance in or on the food.

30. The method of claim 24, wherein the indicator substance and the another indicator substance are embodied in and/or on at least a selected region of the food that is identified as critical, in particular in relation to a cooking point, during a preparation of the food.
